(19) United States
(12) Patent Application Publication
(10) Pub. No.: US 2025/0332073 A1
(43) Pub. Date: Oct. 30, 2025

(54) Oral Iodine Disinfection with Post-Treatment Whitening

(71) Applicant: CAO Group, Inc., West Jordan, UT (US)

(72) Inventor: Steven D Jensen, South Jordan, UT (US)

(73) Assignee: CAO Group, Inc., West Jordan, UT (US)

(21) Appl. No.: 19/187,814

(22) Filed: Apr. 23, 2025

Related U.S. Application Data
(60) Provisional application No. 63/638,318, filed on Apr. 24, 2024.

(51) Int. Cl. A61K 8/04; A61K 8/81; A61Q 11/00

(52) U.S. Cl. CPC A61K 8/042; A61K 8/8176; A61Q 11/00; A61K 2800/48; A61K 2800/74; A61K 2800/884; A61K 2800/92

(57) ABSTRACT

A dental treatment device presents iodine in a solid or semi-solid carrier in order to decrease incidents of accidental swallowing and bolster effective treatment. A whitening treatment is also provided for immediate counteraction of the undesired staining effect of iodine. Treatment is then able to be accomplished in a single setting, whether at home or in a clinical setting.

### CROSS-REFERENCES TO RELATED APPLICATIONS

The present invention claims priority to prior filed U.S. Provisional Application No. 63/638,318 filed on Apr. 24, 2024, and incorporates the same by reference herein in its entirety.

### FIELD OF THE INVENTION

The present invention relates to the field of dentistry and more particularly relates to a non-coalescent gelatinous oral disinfection treatment coupled with a similar whitening treatment to counteract staining.

### BACKGROUND OF THE INVENTION

Some contemporary disinfection treatments utilize iodine solutions to disinfect the oral cavity. These solutions are usually delivered into the mouth, swished around, and thereafter spit out after a standardized amount of treatment time. Polyvinyl pyrrolidone (PVP) has been the preferred thickener of these iodine solutions since it is chemically compatible and provides improved stability with reactive anions, such as iodine (Povidone-iodine). Liquid PVP-iodine solutions are a bit hazardous because of the possibility that a patient might swallow the whole treatment dose instead of spitting it out, especially for children.

Another inherent downside in utilizing iodine as a disinfectant is that it tends to temporarily stain the surface in which it is applied. Iodine usually stains the surface, which would include not only teeth but the soft tissues of the oral cavity, a brown to a brownish orange discoloration. This presents an aesthetic problem when iodine is utilized orally. Most patients would like to maintain their usual appearances and would prefer not to have stained teeth, even if it is a temporary effect.

The present invention is a two-part strategy utilizing 2 non-coalescent gelatinous strips: a PVP-iodine disinfection strip along with a teeth whitening strip. The PVP-iodine strip is applied to teeth for its disinfection properties. The present invention improves upon liquid PVP-iodine solutions by migrating to more “solid type” medication delivery systems, especially highly viscous gels or non-coalescent gelatinous-type flexible films or strips. Since the delivery system is solid, there is less risk of swallowing, and the iodine is concentrated at the locus of treatment rather than evenly distributed about the oral cavity. The purpose of the teeth whitening strip is to quickly remove any residual staining that remains after the PVP-iodine treatment is complete. Therefore, the patient is able to disinfect a desired portion of the oral cavity and bleach away any residual stains or discoloration after the treatment is complete. The present invention represents a departure from the prior art in that the kit of the present invention allows both antimicrobial treatment and whitening treatment to be administered by a patient, practitioner, or other end user while following essentially identical procedures for both treatments.

The teeth whitening strip being comprised of a peroxide-based formulation or any other teeth whitening compound, especially if the pH of the peroxide based whitening strip is greater than 9; and most preferably above 10.

### SUMMARY OF THE INVENTION

In view of the foregoing disadvantages inherent in the known types of oral anti-microbial treatments, an improved treatment kit may provide a treatment methodology that meets the following objectives: that it provides both anti-microbial and whitening treatments in a single kit, that it provides stable active treatment agents to the end user, that the procedures for applying both the anti-microbial treatment and follow-up whitening treatment be easily implemented and essentially identical, that it be cost effective to manufacture, As such, a new and improved treatment kit may comprise two different active formulations, one for anti-microbial treatment and one for teeth whitening, presented in the form of non-coalescent, gelatinous strips which may be applied to oral structures in order to accomplish these objectives.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### DESCRIPTION

A preferred embodiment of the oral treatment kit is herein described. It should be noted that the articles “a”, “an”, and “the”, as used in this specification, include plural referents unless the content clearly dictates otherwise.

The combined method of the present invention incorporates the utilization of both a disinfectant strip and a teeth whitening device in a complete treatment regime. The teeth whitening device may also be non-coalescent strip of a peroxide-based formulation or any other teeth whitening compound, especially if the pH of the peroxide based whitening strip is greater than 9, and most preferably above 10. Reductive formulations may also be used. Suitable devices are described in U.S. Pat. No. 10,646,419 (Mar. 31, 2020), 11,219,582 (Jan. 11, 2022), 11,826,444 (Nov. 28, 2023) and 11,826,445 (Nov. 28, 2023) and U.S. Pub. App. No. 2023/020731 (Jul. 6, 2023), all of these patents and applications being incorporated herein by reference.

Initial construction of the disinfecting PVP-iodine strip involves applying PVP-iodine coalescent gels onto a backing material such as parafilm, polyethylene film, polypropylene film, polyvinyl acetate film, or another flexible water-insoluble backing materials. Then it is subsequently dried into a non-coalescent film layer that is adhered to the backing material forming a PVP-iodine delivery device. During the drying process, the preferred amount of drying produces a non-coalescent flexible gelatinous layer similar to a GUMMI WORM that is able to flex and bend along with the backing material in order to withstand the forces produced while the PVP-iodine delivery device is being applied to the teeth by the patient. The physical manufacture of this embodiment of a disinfectant strip is similar to the manufacture of the teeth whitening strips described in the above incorporated references.

Gel compositions usually comprise a thickener, a solvent or liquid carrier, buffering salts, humectants, sweeteners, and flavoring agents. A possible disinfection gel formulation that would serve as an embodiment of the invention could be:
0.05%—iodine
31.0%—PVP K-90
60.8%—Purified or distilled water
1.0%—Poly acrylic acid
0.3%—Sucralose
0.4%—Peppermint Oil
0.1%—Potassium Hydroxide
3.35%—Glycerin USP
3.0%—Polyethylene glycol M.W. 35,000 These ingredients may be blended together and the pH may be adjusted to 7 by adjusting the final amount of potassium hydroxide. This gel may then be applied to a backing material and dried as explained above into a non-coalescent gelatinous state and then transferred into a packaging for end use.

The PVP-iodine delivery device is designed to be removed from a sealed package by the patient, then released by peeling the film from a release liner thus liberating the delivery film wherein the patient aligns the strip to the teeth and applies the PVP-iodine side of the device to the teeth. The PVP-iodine strip is allowed to remain on the teeth and disinfect until the treatment is complete, thereafter the patient removes the strip by peeling it off the teeth.

The PVP-iodine delivery device of the present invention is removed from a sealed package by the patient. Then released by peeling the film from a release liner thus liberating the delivery film. The patient then aligns the strip to the teeth and applies the PVP-iodine side of the device to the teeth or gums. The PVP-iodine strip is allowed to remain on the teeth and disinfect until the treatment is complete, thereafter the patient removes the strip by peeling it off the teeth. A whitening strip is then removed from the package and applied over the teeth or gums in the same manner as the disinfection strip. The whitening strip is to remain until the discoloration treatment is complete, when it can be removed by peeling it off the teeth. This action completes the entire treatment regime.

Delivery methodologies other than a non-coalescent gel may be employed. Delivery methodologies may include gels loaded into a custom or a one-size-fits-all tray, paint-on-the-teeth solutions, emulsions, gels, resins or varnishes, dry type films or dry type strips, syringe delivered gels, and various other devices utilized to deliver compound to a patient's teeth. When delivering any iodine solution, especially in a tray, it is important that the tray is sealed against the teeth or gums/gingiva such that the iodine solution cannot escape the tray and leak into the mouth. The iodine solution may be delivered to a tray through a tube that is connected to the tray that is sealed onto the teeth or gingiva/gums. After which the Iodine solution is then pressurized in the tubing such that fluid flows through the tubing and into the sealed tray, filling it with iodine solution. Any excess fluid can flow out the tray by means of a return line that is also attached to the tray, such that the fluid in the tray is constantly replenished with new iodine solution.

It is also possible to use polyethyloxazoline (“Oxazoline”) in place of PVP as a thickening agent. Like PVP, Oxazoline is biocompatible and will not react with iodine when a gel is formed and their use is interchangeable in the above incorporated applications and patents. PVP is currently preferred due to regulatory issues; however, should the regulatory environment change or if combinations of Oxazoline and PVP receive approval from relevant government agencies, then Oxazoline may be used in place of PVP in similar fashion.

Although the present invention has been described with reference to preferred embodiments, numerous modifications and variations can be made and still the result will come within the scope of the invention. The described embodiments are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

What is claimed is:

1. An oral disinfection delivery device comprising:
a PVP-iodine coalescent gel composition; and,
a flexible water-insoluble backing material forming a film layer onto which the PVP-iodine coalescent gel composition is applied and thereafter the film is dried until the film is a flexible non-coalescent gelatinous disinfection strip and adhered to the backing material as a gelatinous solid.

2. The oral disinfection delivery device of claim 1, further comprising a teeth whitening treatment regime to be applied to teeth after application of the disinfection strip.

3. The oral disinfection delivery device of claim 2, the teeth whitening treatment regime further comprising a whitening strip.

4. The oral disinfection delivery device of claim 3, the teeth whitening strip being non-coalescent and gelatinous.

5. A method of disinfecting at least a portion of the oral cavity, the method comprising:
applying a PVP-iodine disinfection strip to at least one tooth of a dental arch;
maintaining the PVP-iodine disinfection strip on the at least one tooth for a first effective length of time and removing it thereafter; and,
applying a teeth whitening strip to the same at least one tooth of the dental arch and maintaining the teeth whitening strip thereon for a second effective length of time such that residual discoloration that remains from the disinfection treatment is at least partially removed;
wherein both the disinfection strip and the teeth whitening strip are removed from a package and applied to the teeth.

6. The method of claim 5, both the disinfection and teeth whitening strips being non-coalescent and gelatinous.

7. An oral disinfection delivery device comprising:
An iodine coalescent gel composition; and,
a flexible water-insoluble backing material forming a film layer onto which the iodine coalescent gel composition is applied and thereafter the film is dried until the film is a flexible non-coalescent gelatinous disinfection strip and adhered to the backing material as a gelatinous solid;
wherein the iodine coalescent gel is thickened with a polymer selected from the set of polymers consisting of PVP and oxazoline.

8. The oral disinfection delivery device of claim 1, further comprising a teeth whitening treatment regime to be applied to teeth after application of the disinfection strip.

9. The oral disinfection delivery device of claim 2, the teeth whitening treatment regime further comprising a whitening strip.

10. The oral disinfection delivery device of claim 3, the teeth whitening strip being non-coalescent and gelatinous.
